(19) United States
(12) Patent Application Publication
(10) Pub. No.: US 2019/0298161 A1
(43) Pub. Date: Oct. 3, 2019

(54) DEVICE FOR USE IN HYSTEROSCOPY

(71) Applicant: LINA MEDICAL INTERNATIONAL OPERATIONS AG, Root D4 (CH)

(72) Inventor: Jacob Kollerup JENSEN, Hellerup (DK)

(21) Appl. No.: 16/348,840

(22) PCT Filed: Nov. 9, 2017
(86) PCT No.: PCT/EP2017/078757

§ 371 Date: May 9, 2019

(30) Foreign Application Priority Data
Nov. 9, 2016 (DK) PA 2016 70886

(51) Int. Cl. A61B 1/06; A61B 1/05; A61B 1/07; G02B 23/24; G02B 23/26

(52) U.S. Cl. CPC A61B 1/0676; A61B 1/051; A61B 1/0607; A61B 1/303; G02B 23/2423; G02B 23/26; A61B 1/07

(57) ABSTRACT

A device for visualization of internal tissue of a patient's uterus, the device having a control unit, elongated member extending in an axial direction between a proximal end and a distal end, and image capturing tip. The proximal end is connected to the control unit and the distal end is connected to the image capturing tip. The image capturing tip communicates electric video signals with a monitor. At least the distal end of the elongated member and image capturing tip are dimensioned for insertion into the patient's uterus through cervix. The image capturing tip has a housing, digital imaging device, lens, and at least one light source. To provide slim design and improved capability for insertion into the uterus, the digital imaging device is arranged between the light source and lens, and has a light guide configured to direct light from the light source around the digital imaging device.

### BACKGROUND OF THE INVENTION

### Field of the Invention

The present invention relates to a device for use in hysteroscopy.

### Brief Discussion of the Related Art

Gynaecologists use hysteroscopy in standard examination procedures, both office-based procedures and in hospital procedures where an endoscope is inserted into the uterus e.g. for inspecting the lining.

For the practitioner, the field of diagnostic imaging, for example hysteroscopy, has allowed for the viewing of objects, internal mechanisms, and the like with minimal disruption.

Such imaging tools have been used in a wide variety of settings for detailed inspection, including but not limited to the use and application in the field of medicine.

Since the scope in hysteroscopy is inserted through the narrow passage of the cervix, the existing devices comprises an axially extending, elongated, slim, rod-like member with a small crosswise dimension perpendicular to the axial direction. In traditional scopes, the distal tip contains essentially only a lens, and the image is transferred through the elongated member by fibre optics or glass rods etc. This enables a very slim design and thereby reduces complications related to the insertion into the uterus. In the opposite proximal end, the image is either visualised in an ocular or captured by a digital imaging device such as a CCD (Charge-coupled device).

### SUMMARY OF THE INVENTION

It is an object to provide an improved device for hysteroscopy particularly relative to manoeuvrability and the ability to insert the elongated member into the uterus.

Accordingly, the invention, in a first aspect, provides a device for visualization of internal tissue of a patient's uterus, the device comprising a control unit, an elongated member extending in an axial direction between a proximal end and a distal end, and an image capturing tip, the proximal end being connected to the control unit and the distal end being connected to the image capturing tip, where the image capturing tip is configured to communicate electric video signals with a monitor

The image capturing tip comprises a digital imaging device, a lens, and a light source, wherein the a digital imaging device is arranged between the a light source and the lens, and the device further comprises a light guide configured to direct light from the light source around the digital imaging device.

Herein, the feature of the light guide being configured to direct light from the light source around the digital imaging device means that the light is directed from one side of the digital imaging device to an opposite side of the digital imaging device.

Since the imaging device is arranged between the light source and the lens, the light source becomes located behind the digital imaging device during insertion of the image capturing tip into the uterus, and the light source does therefore not increase the crosswise dimension perpendicular to the axial direction and an easier and/or a more comfortable insertion into the uterus can be facilitated.

At least the distal end of the elongated member and the image capturing tip are dimensioned for insertion into the patient's uterus through cervix. For that purpose, the cross section may particularly have a largest dimension of 5 mm. In one embodiment, the elongated member and/or the image capturing tip have a circular shape with a diameter below 5 mm. such as 4 mm, or even below 4 mm. This is suitable for inspection of a human uterus through cervix.

By definition, the axial direction herein specifies the direction along the centre of the elongated member. The elongated member may follow a straight line, or it may be curved.

The control unit may particularly be dimensioned and shaped to be held by a user's hand and it may include various components such as a monitor, a battery, a fluid flushing system, and other parts which are suitable for the procedure. In that way, the device may form a complete, independent, portable hysteroscopy device, e.g. suitable for single use.

The image capturing tip may include a flow structure for release of fluid from the image capturing tip and for entering fluid into the elongated member via the image capturing tip.

The elongated member may form a tool conduit extending from an inlet at the hand-held control unit to a distal tool opening at or in the image capturing tip. The tool conduit may allow insertion of a surgical tool through the elongated member. The light source and the digital imaging device may particularly be radially offset from the tool conduit such that a tool which is inserted through the tool conduit can pass the digital imaging device and the light source.

The light source may e.g. be located such that it can be projected in the axial direction onto a rear-surface of the digital imaging device, i.e. such that it is completely behind the digital imaging device in the direction of insertion into the uterus.

The light source may particularly be located such that the light is transmitted in a forward direction towards the control unit, or in a crosswise direction perpendicular to the axial direction. Particularly, the digital imaging device may face in a forward direction away from the control unit for capturing images in front of the device, and the light source may face in an opposite rearward direction towards the control unit to direct the light rearwardly towards the control unit.

By directing the light in an opposite direction relative to the direction in which images are captured, the heating from the LED is less intensive on the CCD. The light emitting firstly in the rearward direction and then through the light guide in the forward direction, provides an increased travel length through the light guide and the diffusion can thereby be increased with a small dimension of the image capturing tip.

The light guide may form a light emitting front surface facing in the forward direction. The front surface may e.g. be a ring-shaped front surface extending about the lens, e.g. a circular light emitting front surface. The light guide may further be configured to emit the light evenly along the circumference of the ring shaped front surface. The ring-shaped front surface may e.g. have an outer diameter being in the range of 1,4-2 times or even up to 3 times the inner diameter of the opening in which the lens is located.

The light guide could be formed by the housing of the image capturing tip or at least a part of the housing of the image capturing tip. The light guide may particular be made from a transparent material, e.g. from glass or from a polymer material such as Acryl or Polycarbonate.

The light guide and the lens may be formed in one piece, e.g. from the same material.

At least a part of the transparent material may be covered with a non transparent material to thereby only release light from specific areas of the light guide, e.g. from the aforementioned light emitting front surface. In one embodiment, only that front surface is not covered by the non transparent materiel. At least a part of the non transparent material could be reflective, e.g. a reflective metal material, e.g. a chrome layer with high refractive index.

The light guide forms an encapsulation of the digital imaging device and the lens. In one embodiment, the light guide forms a liquid tight encapsulation.

The light guide may connect the digital imaging device, the light source, and/or the lens to the elongated member. I.e. the light guide may be connected to the elongated member and to at least one of the lens, the light source, and the digital imaging device. In one example, at least one of the digital imaging device, the light source, and the lens is/are arranged in a mould and the light guide is formed by injection of a liquid material into the mould . In that way, the light guide encapsulates and fixes the position of the light source, lens and/or the digital imaging device.

The light guide may diffuse the light from the light source to thereby increase scattering and/or prevent shadows. The light guide may e.g. have a surface pattern making the light diffuse, or it may include particles diffusing the light when it is transmitted through the light guide.

The digital imaging device may particularly include at least one CCD, and the light source may include at least one LED (light emitting diode). In one embodiment, the CCD and lens are integrated into one single entity forming a camera cube.

Electrical connection between the image capturing tip and the control unit may be established by cables or by a PCB (printed circuit board) comprising electrical conductors for transfer of power and/or image signals from the digital imaging device and the light source.

The light source and the digital image device may both be attached to the same PCB which is used for transmission of power and/or signals between the control unit and the image capturing tip.

The PCB may form a bend portion between the digital image device and the light source such that they can be mounted to the same side of the PCB and subsequently be oriented in opposite direction by bending the PCB.

The PCB could be moulded into the light guide to thereby use the light guide for fixing the PCB with the digital imaging device and light source.

The elongated member and/or the image capturing tip may be made from a material selected from the group consisting of: SEBS, PUR, and EVA.

In a second aspect, the invention provides a method of visualization of internal tissue of a patient's uterus by use of a device according to the first aspect. By use of the device, the surgeon may introduce the image capturing tip with less complication due to the reduced diameter obtained by the location of the light source behind digital imaging device.

### BRIEF DESCRIPTION OF THE DRAWINGS

In the following, embodiments of the invention will be described in further details with reference to the drawing in which:

FIG. 1 illustrates an internal tissue visualization device according to the invention;

FIG. 2 illustrates a cross section of the image capturing tip;

FIGS. 3 and 4 illustrate an S-shaped image capturing tip; and

FIGS. 5 and 6 illustrate a straight image capturing tip.

### DETAILED DESCRIPTION OF THE PREFERRED EMBODIMENTS

It should be understood that the detailed description and specific examples, while indicating embodiments of the invention, are given by way of illustration only, since various changes and modifications within the spirit and scope of the invention will become apparent to those skilled in the art from this detailed description.

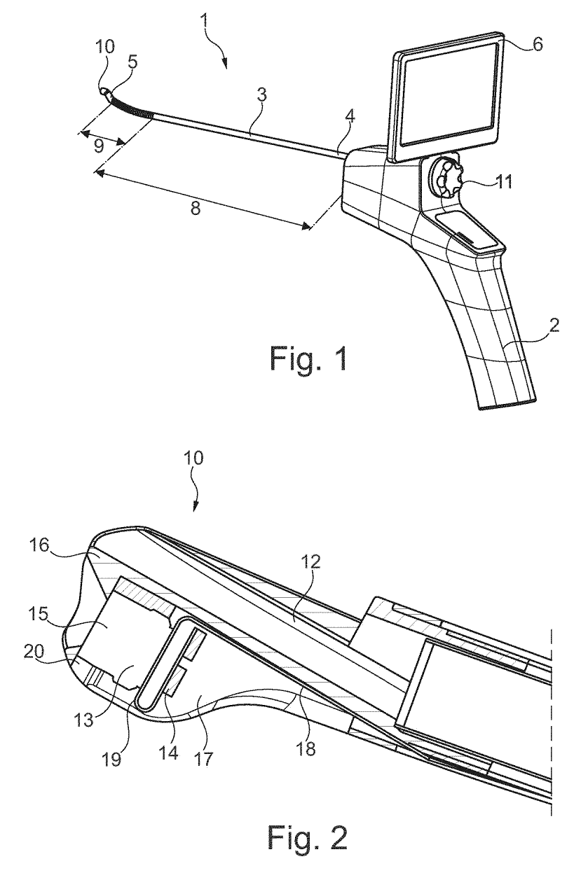

FIG. 1 illustrates a device for tissue visualization device 1 comprises an image capturing structure configured to capture pictures of tissue.

The device 1 comprises a hand held control unit 2 and an elongated member 3 connected to the control unit 2. The elongated member extends from a proximal end 4 to a distal end 5.

The monitor 6 can visualize the captured images. The monitor is attached to the hand held control unit to thereby enable use of the device completely independent on fixtures of an operating room.

The entire elongated member includes a straight portion 8 and a curved portion 9. The curved portion is between the image capturing tip 10 and the straight portion.

The elongated member 3 may be rigid and dimensionally stable to enable insertion through cervix and such that it forms a good support for tools inserted into the uterus through an elongated tool conduit through the elongated member. The rigidity further supports manipulation of the image capturing tip by manipulation of the control unit. The elongated member could e.g. be made of a rigid material such as metal or plastic. The elongated member 3 is relatively long and slim to reduce discomfort for the patient during insertion. The elongated tool conduit may be used for inserting tools such as a scissor, a forceps or a morcellator etc.

The elongated member may e.g. comprise an inner tube and an outer tube. The inner tube is configured to rotate inside the outer tube and comprises a rigid inner section axially coextending a flexible outer section of the outer tube. The rigid inner section may have a curvature and due to its rigidity, it deforms the flexible outer section and thereby forms the curvature of the curved portion 9 by deflection of the flexible outer section.

The control knob 11 is fixed to the inner tube and thereby enables a user to rotate the inner tube by rotation of the control knob and thereby change the orientation of the image capturing tip 10.

The inner conduit within the inner tube allows fluid to be introduced into the uterus during surgery. Such fluids may typically be injected to expand the uterus during a medical procedure, or it may be injected to flush the image capturing structure and thereby create a clear sight. The fluid from the uterus is allowed to drain through the outer conduit between the inner tube and the outer tube. By this structure, fluid may constantly be circulated in the uterus and provide good visibility.

The control unit 2 has the shape of a handle adapted to fit in the hand of the user.

The image capturing tip may have different shapes and will be explained in further details with reference to FIGS. 2-4.

In FIG. 2, the image capturing tip extends along a straight line as an extension of the elongated member. The image capturing tip forms a tool conduit 12 in extension to the tool conduit in the elongated member. The digital imaging device 13, the light source 14, and lens 15 is fixed in a housing 16. The housing holds a light guide 17 made of a transparent plastic material. The light guide directs the rearward light from the light source 14 in an opposite forward direction passed the digital imaging device 13.

The digital imaging device and the light source are both attached to, and wired through the elongated member by the PCB 18 which forms a folded portion 19.

The light guide forms a ring-shaped front surface 20 extending about the lens 15 and being offset into the housing 20.

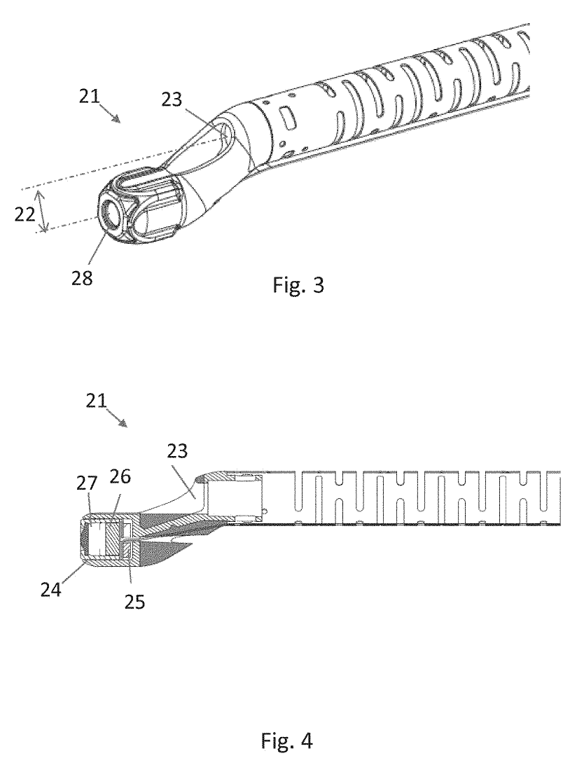

FIGS. 3 and 4 illustrate an S-shaped image capturing tip 21. Due to the S-shape, the image capturing structure including the digital imaging device, the lens, and the light source becomes off-set by the distance indicated by the arrow 22 from the tool conduit 23. A tool which is in the elongated conduit may therefore pass the image capturing structure unhindered. The S-shaped image capturing tip includes a light guide 24 guiding the rearward light from the LED light source 25 in the forward direction passed the CCD and lens 26, 27.

The light guide 24 forms a ring-shaped front surface 28.

The light sources illustrated in FIGS. 2 and 4 are in the form of a double LED structure with two LEDs. Any number of LEDs could be used, or alternative sources, e.g. including traditional light bulbs could be used.

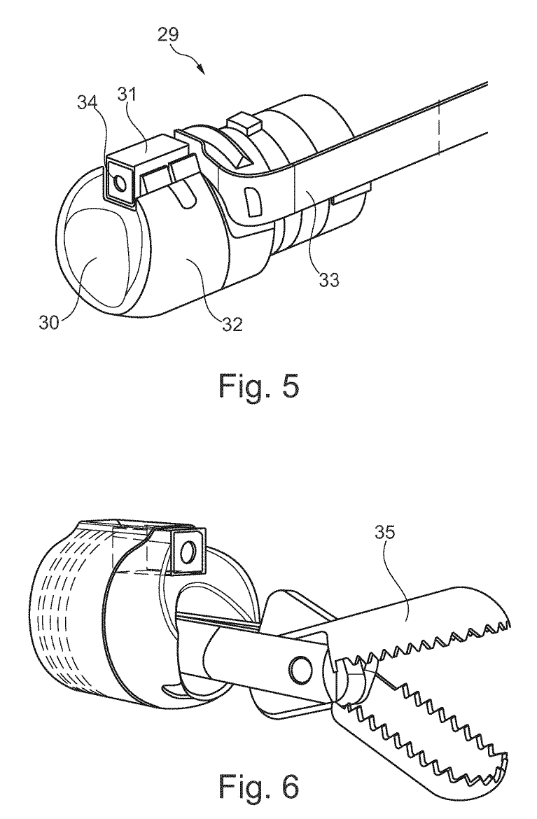

FIGS. 5 and 6 illustrate a straight image capturing tip 29 where the tool conduit 30 and the image capturing structure in the form of the CCD 31 are located within the dimension of the cross section of the elongated member. By this is meant that the image capturing tip can be projected onto a plane which is perpendicular to the distal end of the elongated member and thereby be within the dimension of the elongated member or at most have an area being 10 percent larger than the elongated member. Due to the straight shape, the image capturing tip is can be inserted more easily into the uterus through the relatively narrow cervix.

The straight image capturing tip includes a light guide formed by the body 32 molded about CCD 31 and the PCB 33. The PCB includes at least one rearward facing LED (not shown), and the body 32 guides the rearward light from the LED in the forward direction passed the CCD 31 and lens 34.

The tool 35, in this case a forceps, which is in the elongated conduit may pass the image capturing structure unhindered due to the off-set between the image capturing structure and the tool conduit, and the offset is enabled i.e., due to the rearward LED.

1. A device for visualization of internal tissue of a patient's uterus, the device comprising a control unit, an elongated member extending in an axial direction between a proximal end and a distal end, and an image capturing tip, the proximal end being connected to the control unit and the distal end being connected to the image capturing tip, where the image capturing tip is configured to communicate electric video signals with a monitor, the image capturing tip comprising a digital imaging device, a lens, and a light source, wherein the digital imaging device is arranged between the light source and the lens, and the device further comprises a light guide configured to direct light from the light source around the digital imaging device.

2. The device according to claim 1, wherein the digital imaging device faces in a forward direction away from the control unit, and the light source faces in an opposite rearward direction towards the control unit to transmit light rearwardly towards the control unit.

3. The device according to claim 2, wherein the light guide forms a light emitting front surface facing in the forward direction.

4. The device according to claim 3, wherein the light emitting front surface is ring shaped and extends about the lens.

5. The device according to claim 1, wherein the light guide is formed by a transparent material.

6. The device according to claim 5, wherein at least a part of the transparent material is covered with a non transparent material.

7. The device according to claim 6, wherein at least a part of the non transparent material is a reflective metal material.

8. the device according to claim 1, wherein the light guide forms an encapsulation of at least one of the digital imaging device and the lens.

9. The device according to claim 1, wherein the light guide connects at least one of the digital imaging device and the lens to the elongated member.

10. The device according to claim 1, wherein the light guide diffuses the light from the light source.

11. The device according to claim 1, wherein the digital imaging device comprises a CCD.

12. The device according to claim 1, wherein the light source comprises at least one LED.

13. The device according to claim 1, wherein the light source and the digital image device are attached to the same PCB.

14. The device according to claim 13, wherein the PCB forms a folded portion between the digital image device and the light source.

15. The device according to claim 14, wherein the PCB is moulded into the light guide.

16. A method for visualization of internal tissue of a patient's uterus by use of the device according to claim 1.
